(19) United States
(12) Patent Application Publication
(10) Pub. No.: US 2023/0364357 A1
(43) Pub. Date: Nov. 16, 2023

(54) Heating Units

(71) Applicant: Alexza Pharmaceuticals, Inc., Fremont, CA (US)

(72) Inventors: Krishnamohan SHARMA, Fremont, CA (US); Mingzu LEI, Fremont, CA (US); Karen WANG, Fremont, CA (US); Hoi Sze LAU, Fremont, CA (US); Ron L. HALE, Fremont, CA (US); Reynaldo QUINTANA, Fremont, CA (US); Pravin SONI, Fremont, CA (US)

(21) Appl. No.: 18/223,475

(22) Filed: Jul. 18, 2023

Related U.S. Application Data
(62) Division of application No. 12/211,247, filed on Sep. 16, 2008.

(51) Int. Cl. A61M 11/04; A61M 15/00

(52) U.S. Cl. CPC A61M 11/041; A61M 15/0003; A61M 11/047; A61M 31/00

(57) ABSTRACT

A heating unit comprises a substrate having a first surface and a second surface. A chemical reactant material capable of undergoing an exothermic reaction is disposed on at least a portion of the first surface of the substrate. An igniter is in proximity with the chemical reactant material. A layer of adhesive material overlays at least a portion of at least one of the chemical reactant material and the first surface of the substrate. Other embodiments of the heating unit include a first and a second substrate, each having first and second surfaces positioned with the first surfaces opposing each other in a sandwich construction. A chemical reactant material is disposed on at least a portion of the first surface of at least one of the substrates. The first and the second substrates are sealed together to define a cavity containing the chemical reactant material and an igniter is provided in proximity with the chemical reactant material.

### RELATED APPLICATIONS

This application is a divisional of U.S. application Ser. No. 12/211,247, filed Sep. 16, 2008, the entire disclosure of which is hereby incorporated by reference. Any disclaimer that may have occurred during the prosecution of the above-referenced applications is hereby expressly rescinded, and reconsideration of all relevant art is respectfully requested.

### TECHNICAL FIELD

The present invention pertains to heating units and uses therefor. Such heating units can be employed in a variety of applications, for example, in the delivery of therapeutically effective agents by inhalation.

### BACKGROUND

Pulmonary delivery is known as an effective way to administer physiologically active compounds to a patient for the treatment of diseases and disorders. Devices developed for pulmonary delivery generate an aerosol of a physiologically active compound that can be inhaled by a patient, where the compound can be used to treat conditions in a patient's respiratory tract and/or enter the patient's systemic circulation. Devices for generating aerosols of physiologically active compounds include nebulizers, pressurized metered-dose inhalers, and dry powder inhalers. Nebulizers are based on atomization of liquid drug solutions, while pressurized metered-dose inhalers and dry powder inhalers are based on suspension and dispersion of dry powder in an airflow.

Aerosols for inhalation of physiologically active compounds can also be formed by vaporizing a substance to produce a condensation aerosol comprising the active compounds in an airflow. A condensation aerosol is formed, for example, when a gas phase substance condenses to form particulates. Examples of devices and methods employing vaporization methods to produce condensation aerosols are disclosed in U.S. application Ser. No. 10/861,554, entitled “Multiple Dose Condensation Aerosol Devices and Methods of Forming Condensation Aerosols,” filed Jun. 3, 2004, and U.S. application Ser. No. 10/850,895, entitled “Self-Contained Heating Unit and Drug-Supply Unit Employing Same,” filed May 20, 2004, each of which is incorporated herein by reference in its entirety.

Efficient production of a condensation aerosol comprising a drug is facilitated by rapidly vaporizing the drug such that there is minimal degradation of the drug. The vaporized drug can condense to produce an aerosol characterized by high purity. A secondary benefit is the production of an aerosol in high yield. For use in medical devices, it is useful that the heat source for vaporizing the drug be compact and capable of producing a rapid heat impulse. A variety of heat sources for such devices have been described in the literature.

For example, chemically based heating units, which can include a chemical reactant which is capable of undergoing an exothermic metal oxidation-reduction reaction within an enclosure, are described, for example, in U.S. application Ser. No. 10/850,895, entitled “Self-Contained Heating Unit and Drug-Supply Unit Employing Same,” filed May 20, 2004, incorporated by reference herein in its entirety.

A reactant can be ignited to generate a self-sustaining oxidation-reduction reaction. Once a portion of the reactant is ignited, the heat generated by the oxidation-reduction reaction can ignite adjacent unburned reactant until all of the reactant is consumed in the process of the chemical reaction. The exothermic oxidation-reduction reaction can be initiated by the application of energy to at least a portion of the reactant. Energy absorbed by the reactant or by an element in contact with the reactant can be converted to heat. When the reactant becomes heated to a temperature above the auto-ignition temperature of the reactants (i.e., the minimum temperature required to initiate or cause self-sustaining combustion in the absence of a combustion source or flame), the oxidation-reduction reaction will initiate, igniting the reactant material in a self-sustaining reaction until the reactant is consumed.

As recognized by those of skill in the art, other approaches have also been employed for providing a controlled amount of heat to a drug delivery device, for example, using electrochemical interactions. Here, components that interact electrochemically after initiation in an exothermic reaction are used to generate heat. Exothermic electrochemical reactions include reactions of a metallic agent and an electrolyte, such as a mixture of magnesium granules and iron particles as the metallic agent, and granular potassium chloride crystals as the electrolyte. In the presence of water, heat is generated by the exothermic hydroxylation of magnesium, where the rate of hydroxylation is accelerated in a controlled manner by the electrochemical interaction between magnesium and iron, which is initiated when the potassium chloride electrolyte dissociates upon contact with the liquid water. Electrochemical interactions have been used, for example, in the smoking industry to volatilize tobacco for inhalation (U.S. Pat. Nos. 5,285,798; 4,941,483; 5,593,792).

The aforementioned self-heating methods are capable of generating heat sufficient to heat an adjacent article to several hundred degrees Celsius in a period of several minutes. However, there remains a need in the art for compact, convenient devices that are capable of rapid heat production, that is, on the order of seconds and fractions of seconds, and that are also capable of heating an article to within a defined temperature range, and which devices are also suitable for use in articles for human use.

### SUMMARY

Novel heating units, and uses therefor, are disclosed. The heating units have many advantages over prior art heating units. The heating units disclosed herein are compact, provide substantially uniform temperature distribution across the surface of the device, have excellent handling properties and shelf life, are readily and inexpensively prepared from readily available starting materials, can be coated with a wide variety of physiologically active compounds for delivery of a wide range of doses thereof, and can be safely disposed after use because no toxic chemicals are employed in the preparation thereof.

Heating units in accordance with the invention can be used for the delivery of a wide range of physiologically active compounds by a preferred mode of delivery, for example, by inhalation.

One embodiment disclosed herein is a heating unit comprising: a substrate having a first surface and a second surface; a chemical reactant material capable of undergoing an exothermic reaction disposed upon at least a portion of the first surface of the substrate; an igniter in proximity with the chemical reactant material; and a layer of an adhesive material overlying at least a portion of one of the chemical reactant material or the first surface of the substrate. (As used herein, the term “proximity” refers to an igniter that is positioned relative to the chemical reactant material to ignite it upon actuation of the igniter. For example, it may be directly in contact with the chemical reactant material or disposed within a distance of 500 μm or less from the chemical reactant material or within some other distance wherein the igniter can ignite the chemical reactant material upon actuation.)

The adhesive layer may also overlay at least a portion of the reactant material, and the adhesive material may be compatible with the reactant material. (As used herein, the term “compatible” refers to an adhesive material that is substantially non-reactive with the reactant material.)

The heating unit may further comprise a second substrate having a first surface and a second surface. The first surface of the second substrate may be in contact with the adhesive layer. Alternatively, a chemical reactant material capable of undergoing an exothermic reaction may be disposed on at least a portion of the first surface of the second substrate, and the chemical reactant material may be in contact with the adhesive layer. In a particular embodiment, the first and second substrates are part of a single component, folded so as to form a unitary structure containing the reactant material within and, optionally, sealed.

In accordance with an alternative embodiment, disclosed herein is a heating unit comprising: a first substrate and a second substrate, where each of the first and second substrates have a first surface and a second surface; a chemical reactant material capable of undergoing an exothermic reaction disposed upon at least a portion of the first surface of at least one of the substrates; and an igniter in proximity with the chemical reactant material.

The first substrate and the second substrate may be sealed together. In one embodiment, they are hermetically sealed together. The first and second substrates may be sealed together using any one or more of a number of methods known in the art, such as, for example and not by way of limitation, adhesive sealing, seam welding, spot welding, ultrasonic welding, crimping, and molding, with adhesive sealing and seam welding being presently preferred. The adhesive material may be an inorganic adhesive, an organic adhesive, or an organic/inorganic composite adhesive. Ceramic adhesives have been advantageous. The adhesive material may be a pressure-sensitive adhesive or a hot-melt glue adhesive. The adhesive material is typically in contact with at least a portion of the first surface of both the first and second substrates. The adhesive material may not be in contact with the chemical reactant material.

A chemical reactant material capable of undergoing an exothermic reaction may be disposed on at least a portion of the first surface of one or both substrates. In a particular embodiment, the first and second substrates are part of a single component, folded so as to form a unitary structure containing the reactant material within.

Also contemplated herein are aerosol drug delivery devices comprising the heating units. One embodiment is a multi-dose aerosol drug delivery device comprising a plurality of such heating units.

The substrate may comprise a glass, a ceramic, or a metal foil, such as a steel foil or an aluminum foil. In embodiments of the invention in which there are two substrates, the individual substrates may comprise either the same material or different materials.

The chemical reactant material may comprise a metal reducing agent and a metal-containing oxidizing agent. The metal reducing agent may be selected from the group consisting of molybdenum, magnesium, calcium, strontium, barium, boron, titanium, zirconium, vanadium, niobium, tantalum, chromium, tungsten, manganese, iron, cobalt, nickel, copper, zinc, cadmium, tin, antimony, bismuth, aluminum, and silicon. The metal-containing oxidizing agent may be selected from the group consisting of transition metal oxides, lanthanide metal oxides, and mixed metal oxides. More particularly, the metal-containing oxidizing agent may be a transition metal oxide selected from the group consisting of oxides of iron, copper, cobalt, molybdenum, vanadium, chromium, manganese, silver, tungsten, magnesium, and niobium, for example and without limitation.

The chemical reactant material optionally further comprises a binding agent, which may be selected from the group consisting of clays, metal silicates, phosphate-containing materials, alkoxides, metal oxides, inorganic polyanions, inorganic polycations, inorganic sol-gel materials, synthetic ion exchange resins, zeolites, and diatomaceous earth.

Examples of chemical reactant materials for use in the invention include Zr:Fe₂O₃, Zr:Fe₂O₃:MnO₂, Zr:CuO, Zr:Co₂O₃, Zr:Co₃O₄, and Zr:MoO₃. In one embodiment, the reactant material further includes an amount of a Laponite® additive (a synthetic layered silicate manufactured by Rockwood Additives Limited, Widnes, United Kingdom, and available from Southern Clay Products, Inc., Gonzales, TX).

The chemical reactant material may be printed as lines or patches onto the first surface of the substrate. This may increase the contact/binding area between the substrate surface and the adhesive material, and thereby enhance the rigidity of the adhesive layer during or after ignition.

The heating units may further comprise an igniter in proximity with the chemical reactant material, for the purpose of igniting the chemical reactant material. The igniter may be an optical igniter, a percussive igniter, or an electrical igniter, for example and not by way of limitation. Alternatively, the igniter may be a printable igniter of the type described in U.S. patent application Ser. No. ______ (Attorney Docket No. 84.01R), filed on even date herewith. Such an igniter comprises at least two conductors in a spaced-apart configuration, and a conductive layer bridging the at least two conductors. The conductive layer, which is adapted to initiate and produce a “glow” (i.e., localized heat) upon application of electrical power, has an electrical resistance that is greater than the electrical resistance of both of the at least two conductors. Upon initiation of the conductive layer, heat from the exothermic oxidation of the conductive layer composition is generated sufficient to actuate a reactant composition (e.g., a reactant composition-coated substrate).

The adhesive material may comprise an organic-based adhesive, an inorganic-based adhesive, or a hybrid organic-inorganic-based adhesive. An inorganic-based adhesive has been effective. The inorganic-based adhesive may comprise a ceramic selected from the group consisting of alumina, magnesia, zirconia, silica, metal nitrides, metal silicates, and metal phosphates. In one embodiment, the adhesive material comprises alumina in combination with silica in a ratio of approximately 1:1.

The adhesive material adheres to the chemical reactant material and may have a curing temperature within the range of 60° C. to 400° C. The adhesive material may be in a form such as a ceramic mat, a ceramic block, or a metal foil coated with ceramic adhesive.

An additional layer or layers of material may overlie the adhesive material. The additional layer may comprise a material such as a ceramic adhesive, a polymeric coating (such as an acrylate coating, an epoxy coating, or a maleimide-based coating, for example and not by way of limitation), an organic/inorganic composite material, and a plastic mold. For example, the additional layer may comprise a ceramic that is either the same or different than the adhesive material. In one embodiment, the adhesive material comprises alumina and the additional layer comprises zirconia.

The heating unit may further include a vaporizable component, typically a drug, coated onto the second surface of the substrate. In embodiments of the invention in which there are two substrates, the vaporizable component may be coated onto the second surface of one or both substrates.

### BRIEF DESCRIPTION OF THE DRAWINGS

FIG. 1A is a cross-sectional side view of one embodiment of a heating unit, where an adhesive layer overlies a chemical reactant material which overlies a surface of a substrate, as well as a portion of the substrate.

FIG. 1B is a cross-sectional side view of an embodiment of the heating unit shown in FIG. 1A, which further comprises a second substrate overlying the adhesive layer.

FIG. 1C is a cross-sectional side view of an alternative embodiment of the heating unit shown in FIG. 1B, which further comprises an additional material layer between the second substrate and the adhesive layer.

FIG. 2A is a cross-sectional side view of an embodiment of a heating unit in which an adhesive material is used to bond a first substrate to a second substrate, forming a sandwich around a chemical reactant material.

FIG. 2B is a cross-sectional side view of an embodiment of the heating unit shown in FIG. 2A, which further comprises an additional material layer adjacent to the adhesive layer.

FIG. 2C is a cross-sectional side view of an embodiment of a heating unit in which seam welding is used to bond a first substrate to a second substrate, forming a sandwich around a chemical reactant material.

FIG. 2D is a cross-sectional side view of an embodiment of a heating unit wherein a first substrate and a second substrate are part of a single component folded together and sealed to form a unitary structure containing reactant material within.

FIG. 3A is a top view of a chemical reactant material printed as a pattern on a surface of a substrate

FIG. 3B is a side view of the heating unit shown in FIG. 3A, with an adhesive layer overlying the patterned reactant layer.

FIG. 4 is a cross-sectional side view of an embodiment of a heating unit which includes an igniter and a drug layer.

### DETAILED DESCRIPTION

We have discovered that the pressures in current aerosol drug delivery devices (such as described in U.S. Pat. No. 7,090,830, and U.S. patent application Ser. No. 10/850,895) can be minimized by placing a thin metallic (e.g., stainless steel) or ceramic object in close contact with the chemical reactant coating or by minimizing or eliminating the amount of air trapped inside the sealed unit (for example, by vacuum sealing the unit). Furthermore, our experimental results indicate that ceramic cements/adhesives/binding agents can be coated over chemical reactant coatings on steel foils and the chemical reactant coatings easily ignited, while retaining the ceramic sealing. These discoveries have opened up numerous possibilities with regard to simple and reliable chemical heating unit design.

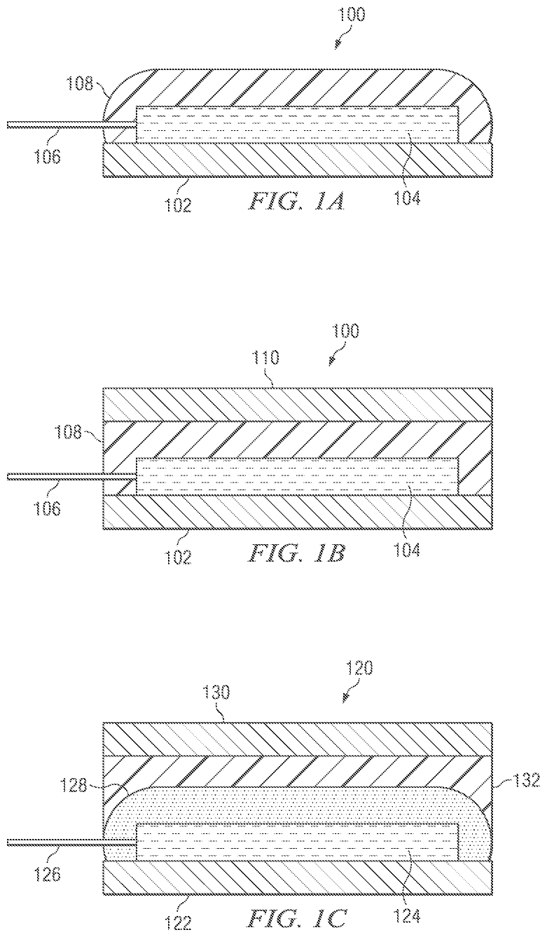

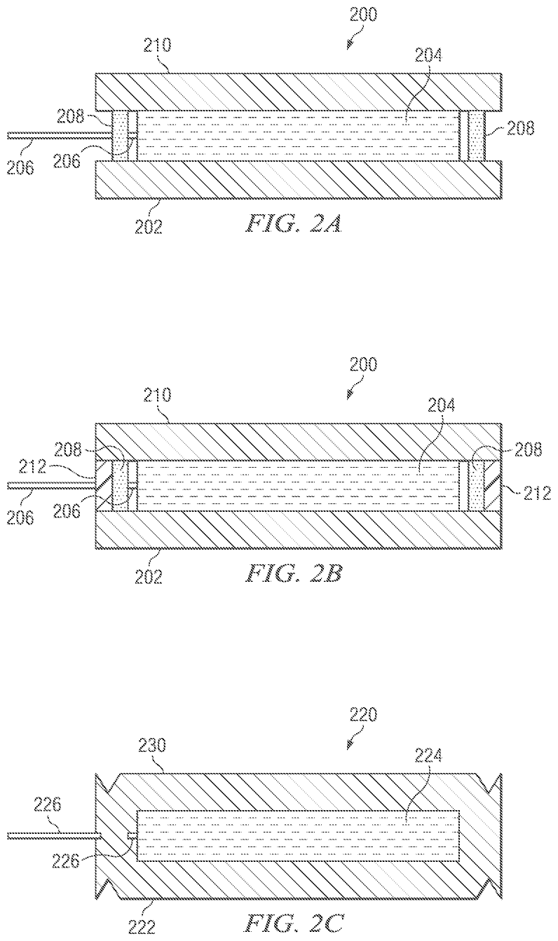

FIGS. 1A-1C and 2A-2D depict various alternative embodiments of a heating unit.

FIG. 1A is a cross-sectional side view of a first embodiment of a heating unit. The heating unit 100 shown in FIG. 1A comprises a substrate 102, with an overlying layer 104 of a chemical reactant material. An igniter 106 is illustrated in contact with the chemical reactant material layer 104. In other embodiments the igniter need only be in sufficient proximity to, upon ignition, ignite the chemical reactant material layer. The chemical reactant material layer 104 is in turn overlaid by an adhesive layer 108.

FIG. 1B is a cross-sectional side view of the heating unit 100 of FIG. 1A, with a second substrate 110 overlying adhesive layer 108.

FIG. 1C is a cross-sectional side view of an alternative embodiment of the heating unit shown in FIG. 1B. The heating unit 120 shown in FIG. 1C comprises a substrate 122, with an overlying layer 124 of a chemical reactant material. An igniter 126 is illustrated in contact with the chemical reactant material layer 124. In other embodiments the igniter need only be in sufficient proximity to, upon ignition, ignite the chemical reactant material layer. The chemical reactant material layer 124 is in turn overlaid by an adhesive layer 128, which is further overlaid by an additional material layer 132. A second substrate 130 overlies additional material layer 132. The adhesive layer 128 may comprise a ceramic adhesive, and the additional material layer 132 may comprise an epoxy adhesive, to provide hermetic sealing of the heating unit 120.

FIG. 2A is a cross-sectional side view of an embodiment of a heating unit in which an adhesive material is used to bond a first substrate to a second substrate, forming a sandwich around a chemical reactant material. The heating unit 200 shown in FIG. 2A comprises a first substrate 202, with an overlying layer 204 of a chemical reactant material. An igniter 206 is shown in contact with the chemical reactant material layer 204. In other embodiments the igniter need only be in sufficient proximity to, upon ignition, ignite the chemical reactant material layer. A second substrate 210 overlies chemical reactant material layer 204. An adhesive material 208 is disposed around the edges of the first substrate 202, but the adhesive material 208 does not contact the chemical reactant material layer 204. The adhesive material 208 is in contact with both the first substrate 202 and the second substrate 210, bonding the two substrates together to form a sealed sandwich around the chemical reactant material layer 204.

FIG. 2B is a cross-sectional side view of an embodiment of the heating unit shown in FIG. 2A, which further comprises an additional material layer adjacent to the adhesive layer. FIG. 2B shows the heating unit 200 of FIG. 2A, with an additional material 212 adjacent to adhesive material 208. In one embodiment, the adhesive material 208 comprises a ceramic adhesive, and the additional material 212 comprises an epoxy adhesive, to provide hermetic sealing of the heating unit 200.

FIG. 2C is a cross-sectional side view of an embodiment of a heating unit in which seam welding is used to bond a first substrate to a second substrate, forming a sandwich around a chemical reactant material. The heating unit 220 shown in FIG. 2C comprises a first substrate 222, with an overlying layer 224 of a chemical reactant material. An igniter 226 is illustrated in contact with the chemical reactant material layer 224. In other embodiments the igniter need only be in sufficient proximity to, upon ignition, ignite the chemical reactant material layer. A second substrate 230 overlies chemical reactant material layer 224. The first substrate 222 and the second substrate 230 are seam welded together to form a sandwich around the chemical reactant material layer 224.

FIG. 2D is a cross-sectional side view of an embodiment of a heating unit 240 comprising a single substrate 242 folded over itself with a chemical reactant material layer 244 deposited on opposing surfaces of the folded over substrate 242. An igniter 226 is illustrated in contact with the chemical reactant material layers 244. In other embodiments the igniter need only be in sufficient proximity to, upon ignition, ignite the chemical reactant material layer. The opposing edges of the substrate 242 are seam welded to seal the chemical reactant material layers 244 within the substrate 242, defining a unitary body structure containing the reactant material within.

One skilled in the art to which the invention belongs can envision alternative embodiments beyond the basic embodiments of the heating units depicted in FIGS. 1A-1C and 2A-2D.

Descriptions and examples of each of the various layers and/or components of heating units in accordance with the invention are provided below.

Substrates

A variety of substrates are contemplated for use in heating units according to the invention. Substrate materials include metals, metal alloys, and ceramics (including glasses).

Presently preferred substrates are thin to facilitate heat transfer from the interior to the exterior surface and/or to minimize the thermal mass of the device. In certain embodiments, the substrate has a thickness in the range of 0.001 inch to 0.020 inch; in other embodiments, in the range of 0.001 inch to 0.010 inch; more typically, in the range of 0.002 inch to 0.006 inch; and, in yet other embodiments, in the range of 0.002 inch to 0.005 inch.

In certain embodiments, a thin substrate can facilitate rapid and homogeneous heating of the exterior surface with a lesser amount of reactant material compared to a thicker substrate. The substrate can also provide structural support for the reactant material and an optional material to be heated, such as for example, a drug film.

A presently preferred substrate is a metal foil. Examples of metal foils include stainless steel, copper, aluminum, and nickel, as well as alloys thereof.

Alternatively, the substrate may comprise a ceramic. As used herein, the term “ceramic” refers to complex compounds and solid solutions of both metallic and nonmetallic elements joined by ionic and covalent bonds. Ceramic materials may be a combination of inorganic elements, although they may contain carbon. Examples of ceramic materials include, but are not limited to, metallic oxides (such as oxides of aluminium, silicon, magnesium, zirconium, titanium, chromium, lanthanum, yttrium, and mixtures thereof) and non-oxide compounds including, but not limited to, carbides (such as carbides of titanium, tungsten, boron, silicon, and mixtures thereof), silicides (such as molybdenum disicilicide), nitrides (such as nitrides of boron, aluminium, titanium, silicon, and mixtures thereof) and borides (such as borides of tungsten, titanium, uranium, and mixtures thereof), and mixtures thereof; spinels, titanates (such as barium titanate, lead titanate, lead zirconium titanate, strontium titanate, iron titanate), ceramic super conductors, zeolites, ceramic solid ionic conductors (such as yittria-stabilized zirconia, beta-alumina, and cerates).

Substrates can have one or more layers, and the multiple layers can comprise different materials. For example, a substrate can comprise multiple layers of laminated metal foils, and/or can comprise thin films of one or more materials deposited on the surface. The multiple layers can be used for example to determine the thermal properties of the substrate and/or can be used to determine the reactivity of the surface with respect to a compound disposed on the exterior surface thereof. A multilayer substrate can have regions comprising different materials.

Heating units according to the present invention may also further include a second substrate having a first surface and a second surface. The second substrate may be incorporated into the heating unit so as to provide a “sandwich”-like structure, where the resulting structure includes a first substrate having a first surface and a second surface, at least one reactant material disposed upon a portion of the first surface of the first substrate, where the reactant material is capable of undergoing an exothermic reaction, at least one adhesive layer disposed upon at least a portion of the reactant material and/or the substrate, and a second substrate having a first and second surface, disposed opposite the first surface of the first substrate.

Alternatively, heating units of the invention can be configured such that the first and second substrates are part of a single component which can be folded to form a unitary structure having the reactant material contained within. Upon folding the first and second substrate materials together, they can be sealed (for example, by use of adhesive, crimping, or welding) so as to form a highly stable heating device. One such embodiment is illustrated in FIG. 2D.

One of the many advantages of the heating units described herein is the sizable surface area thereof for the application of one or more vaporizable components (or multiple doses of the same vaporizable component) thereto. Heating units can be prepared from substrates having surface areas of at least 0.2 cm², with surface areas within the range of 0.2 cm² to 50 cm² per heating unit being desirable.

The heating units described herein can be configured to comprise multiple sources of reactant material associated with a substrate surface area.

As used herein, the term “surface area per heating unit” can refer to the surface area associated with a single source or multiple sources of reactant material. As used herein, the term “surface area per heating device” refers to the total surface area associated with all sources of reactant material in a heating device, which may include multiple heating units.

Another advantage of the heating units of the invention is their relatively small dimensions. For example, the heating units can be prepared to have a thickness of 10 mm or less, with thicknesses as low as 0.04 mm being possible. The thinness of the heating units allows multiple units to be stacked on top of each other to increase the heated surface area or in one embodiment to deliver multiple doses from a smaller inhalation drug delivery device.

### Chemical Reactant Materials

Chemical reactant materials contemplated for use in the practice of the present invention are available in many forms such as, for example and not by way of limitation, solids, gels, liquids, and combinations thereof. Such materials can achieve an exothermic reaction in a variety of ways, for example, by means of a metal oxidation-reduction reaction or an intermetallic alloying reaction.

An oxidation-reduction reaction refers to a chemical reaction in which one compound gains electrons and another compound loses electrons. The compound that gains electrons is referred to as an oxidizing agent, and the compound that loses electrons is referred to as a reducing agent. An example of an oxidation-reduction reaction is a chemical reaction of a compound with molecular oxygen (O₂) or an oxygen-containing compound that adds one or more oxygen atoms to the compound being oxidized. During the oxidation-reduction reaction, the molecular oxygen or the oxygen-containing compound is reduced by the compound being oxidized. The compound providing oxygen acts as the oxidizer or oxidizing agent. The compound being oxidized acts as the reducing agent. Oxidation-reduction reactions can be exothermic, meaning that the reactions generate heat. An example of an exothermic oxidation-reduction reaction is the thermite reaction of a metal with a metal-containing oxidizing agent. In certain embodiments, reactant material can comprise a metal reducing agent and an oxidizing agent such as, for example and not by way of limitation, a metal-containing oxidizing agent.

In certain embodiments, a metal reducing agent can include, but is not limited to, molybdenum, magnesium, calcium, strontium, barium, boron, titanium, zirconium, vanadium, niobium, tantalum, chromium, tungsten, manganese, iron, cobalt, nickel, copper, zinc, cadmium, tin, antimony, bismuth, aluminum, and silicon. In certain embodiments, a metal reducing agent can include aluminum, zirconium, and titanium. In certain embodiments, a metal reducing agent can comprise more than one metal reducing agent.

In certain embodiments, an oxidizing agent can comprise oxygen, an oxygen-based gas, and/or a solid oxidizing agent. In certain embodiments, an oxidizing agent can comprise a metal-containing oxidizing agent. In certain embodiments, the metal-containing oxidizing agent is selected from the group consisting of transition metal oxides, lanthanide metal oxides, and mixed metal oxides. For example, the metal-containing oxidizing agent may be a transition metal oxide selected from the group consisting of oxides of iron (e.g., Fe₂O₃), copper (e.g., CuO), cobalt (e.g., CO₃O₄), molybdenum (e.g., MoO₃), vanadium (e.g., V₂O₅), chromium (e.g., CrO₃, Cr₂O₃), manganese (e.g., MnO₂), silver (e.g., Ag₂O), tungsten (e.g., WO₃), (e.g., MgO), and niobium (e.g., Nb₂O₅), for example and without limitation. In certain embodiments, the metal-containing oxidizing agent can include more than one metal-containing oxidizing agent.

In certain embodiments, the metal reducing agent forming the reactant material can be selected from zirconium and aluminum, and the metal-containing oxidizing agent can be selected from MoO₃, Fe₂O₃, and MnO₂.

The ratio of metal reducing agent to metal-containing oxidizing agent can be selected to determine the ignition temperature and the burn characteristics of the reactant material. For example, a chemical reactant can comprise 75% zirconium and 25% MoO₃, percentage based on weight. In certain embodiments, the amount of metal reducing agent can range from 60% to 90% of the total dry weight of the reactant material. In certain embodiments, the amount of metal-containing oxidizing agent can range from 10% to 40% of the total dry weight of the reactant material.

In certain embodiments, the amount of oxidizing agent in the reactant material can be related to the molar amount of the oxidizers at or near the eutectic point for the reactant composition. In certain embodiments, the oxidizing agent can be the major component and, in others, the metal reducing agent can be the major component. The particle size of the metal and the metal-containing oxidizer can be varied to determine the burn rate, with smaller particle sizes selected for a faster burn (see, for example, U.S. Pat. No. 5,603,350).

In certain embodiments, reactant material can comprise additive materials to facilitate, for example, processing and/or to determine the thermal and temporal characteristics of a heating unit during and following ignition of the reactant material. An additive material can be reactive or inert. An inert additive material will not react or will react to a minimal extent during ignition and burning of the reactant material. Additive materials can be organic or inorganic materials and can function as binding agents, adhesives, gelling agents, thixotropic agents, and/or surfactants. Examples of gelling agents include, but are not limited to, clays such as Laponite® or Cloisite® additives (manufactured by Rockwood Additives Limited, Widnes, United Kingdom, and available from Southern Clay Products, Inc., Gonzales, TX); montmorillonite (a very soft phyllosilicate mineral that typically forms microscopic crystals); metal alkoxides, such as those represented by the formula R—Si(OR)ₙ and M(OR)ₙ, where n can be 3 or 4, and M can be Ti, Zr, Al, B, or another metal; and colloidal particles based on transition metal hydroxides or oxides. Examples of binding agents include, but are not limited to, clays, metal silicates (including soluble silicates such as sodium, potassium, and aluminum silicates), phosphate-containing materials (such as minerals of the phosphate or oxide class), in particular, minerals containing copper, zinc, iron, aluminum, manganese, and titanium, alkoxides, metal oxides, inorganic polyanions, inorganic polycations, and inorganic sol-gel materials, such as alumina or silica-based sols. Binding materials can also include materials such as synthetic ion exchange resins, zeolites (synthetic or naturally occurring), and diatomaceous earth.

In one embodiment, the reactant material further includes a Laponite® additive. Laponite additives are synthetic layered silicates, in particular, magnesium phyllosilicates, with a structure resembling that of the natural clay mineral hectorite (Na_{0.4}Mg_{2.7}Li_{0.3}SiO₁₀(OH)₂). Laponite RD (59.5% SiO₂: 27.5% MgO: 0.8% Li₂O: 2.8% Na₂O) is a commercial grade material which, when added to water, rapidly disperses to form a gel. Laponite RDS (54.5% SiO₂: 26% MgO: 0.8% Li₂O: 5.6% Na₂O: 4.1% P₂O₅) is a commercially available sol-forming grade of Laponite modified with a polyphosphate dispersing agent, or peptizer, to delay rheological activity until the Laponite RDS is added as a dispersion into a formulation. A sol refers to a colloid having a continuous liquid phase in which solid is suspended in a liquid. In the presence of electrolytes, Laponite additives can act as gelling, binding, and/or thixotropic agents. Thixotropy refers to the property of a material to exhibit decreased viscosity under shear.

Presently preferred reactant materials for use herein include Zr:Fe₂O₃, Zr:Fe₂O₃:MnO₂, Zr:CuO, Zr:Co₂O₃, Zr:Co₃O₄, and Zr:MoO₃. For example and not by way of limitation, a typical reactant material for use in the present invention can comprise between about 60 to about 80% Zr, between about 20 to about 40% Fe₂O₃, and between about 1 and about 10% Laponite (in weight percent).

We have found that the addition of an amount of manganese oxide (MnO₂) to the reactant material allows for the peak temperature attained by the substrate (e.g., a steel foil) during heating to be modulated, as disclosed in commonly assigned, copending U.S. patent application Ser. No. ______ (Attorney Docket No. 88.01R), filed on even date herewith. In some embodiments, the reactant material further includes a Laponite® additive.

When incorporated into a reactant material composition comprising a metal reducing agent and a metal-containing oxidizing agent, such as any of those disclosed herein, in addition to imparting gelling and thixotropic properties, Laponite® RDS can also act as binding agent. A binding agent refers to an additive that produces bonding strength in a final product. The binding agent can impart bonding strength, for example, by forming a bridge, film, matrix, and/or chemically self-react and/or react with other constituents of the formulation, preferably imparting added resistance to cracking within the film.

Minimizing the reactant coating thickness can facilitate control of the heating process, as well as facilitate miniaturization of a drug supply unit incorporating a heating unit as described herein. The reactant material may be disposed on the substrate as a thin layer having a thickness within the range of 10 μm to 500 μm; in other embodiments, within the range of 10 μm to 100 μm; in yet other embodiments, within the range of 20 μm to 60 μm.

In certain embodiments, when the reactant material is disposed on the substrate as a film or thin layer, it can be useful that the reactant material adhere to the surface of the substrate and that the constituents of the reactant material adhere to each other and maintain physical integrity. In certain embodiments, it can be useful that the reactant material remain adhered to the substrate surface and maintain physical integrity during processing, storage, and use, during which time the coating of reactant material can be exposed to a variety of mechanical and environmental conditions. Several additives, such as those disclosed herein, can be incorporated into the reactant material to impart adhesion and physical robustness to the coating of reactant material.

By way of example, small amounts of Laponite® RDS added to a slurry of reactant material comprising a metal reducing agent and a metal-containing oxidizing agent can impart thixotropic, gelling, and, in particular, adhesive, properties to the reactant material.

In certain embodiments, the reactant material can comprise a multi-layer comprising reactants capable of undergoing a self-sustaining exothermic reaction. Each of the multiple layers can be homogeneous or heterogeneous. A multi-layer reactant material may comprise alternating and/or interposed layers of materials capable of reacting exothermically. The layers may be continuous or discontinuous. A discontinuous layer refers to a layer that can be patterned and/or a layer that has openings. The use of discontinuous layers can increase the ability to control contact between the reactants and, by bringing the reactants into proximity, can facilitate the exothermic reaction. Each layer can comprise one or more reactants, and can comprise one or more additive materials such as binding agents, gelling agents, thixotropic agents, adhesives, and surfactants.

The reacting layers can be formed into a multi-layer structure by any appropriate method that, at least in part, can be determined by the chemical nature of the reactants in a particular layer. In certain embodiments, metal foils or sheets of two or more reactants can be cold pressed/rolled to form a multi-layer reactant material. Multi-layer reactant materials can comprise alternating or mixed layers of reactants. The layers can be formed, for example and not by way of limitation, by vapor deposition, sputtering, or electrodeposition methods. Using wet coating methods, multiple layers of dispersions comprising the reactants can be deposited to form a multi-layer reactant material, where each layer can comprise the same or different composition.

The number of layers and the thickness of each layer of reactants can be selected to establish the thermal and temporal characteristics of the exothermic reaction. Depending in part on the method used to form the multilayer reactant material, the thickness of a layer can range from, for example, 0.1 μm to 200 μm for a metal sheet, and can range from, for example, 1 nm to 100 μm for a vapor- or electro-deposited layer. The reactant layers can comprise elemental metals, alloys and/or metal oxides. Examples of layer pairs can include, but are not limited to Al:Ni, Al:Cu, Ti:Ni, Ti:C, Zr:B, Mo:Si, Ti:Si, and Zr:S. These and other combinations of reactants and/or additive materials can be used to control the burning characteristics of the reactant material.

In certain embodiments, the multi-layer structure can be repeatedly mechanically deformed to intermix the reactant layers. In certain embodiments, such as where layers are deposited by, for example, vapor deposition, sputtering, or electrodeposition methods, the reactants can be deposited to form an intermixed or heterogeneous composition.

In addition to the layers comprising reactants, a multi-layer reactant material structure can comprise layers of non-reacting materials or materials having certain reaction properties to facilitate control of the thermal and temporal characteristics of the exothermic reaction.

In certain embodiments, a reactant material can be machined, molded, pre-formed, or packed. The reactant material can be formed as a separate element configured to be inserted into a heating unit, or the reactant material can be applied directly to a heating unit. In certain embodiments, reactant material can be coated, applied, or deposited directly onto a substrate forming part of a heating unit, onto a support that can be incorporated into a heating unit, or onto a support configured to transfer the reactant material to a substrate forming a heating unit.

The reactant material can be any appropriate shape and have any appropriate dimensions. For example, reactant material can be shaped for insertion into a square or rectangular heating unit. To increase the contact/binding area between the substrate surface and the overlying adhesive layer, and thereby enhance the rigidity of the adhesive layer during or after ignition, reactant slurry can be printed as lines or patches on the substrate surface. Further, thermally conducting ceramics (such as aluminum nitride) may efficiently transfer heat and promote uniform heating on a substrate (such as a metal foil) surface even if the reactant is deposited as closely spaced lines. A variety of patterns of varying shapes and sizes can be printed onto substrate surfaces.

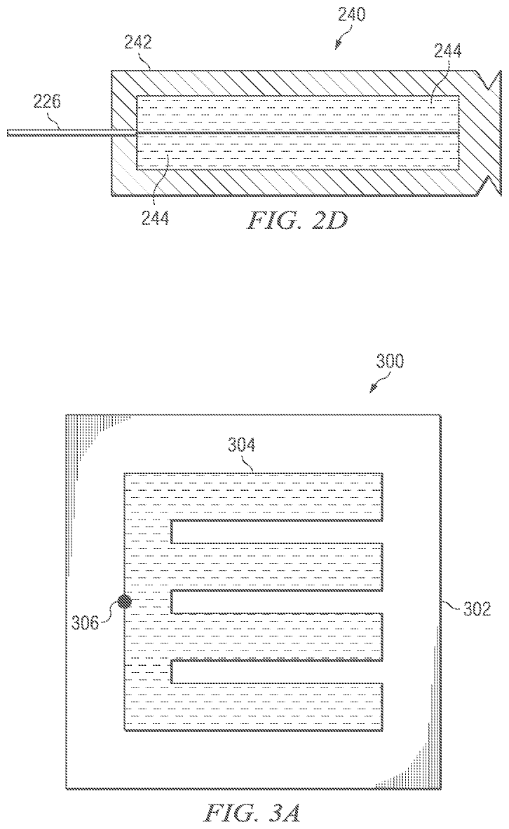

FIG. 3A shows a top view of a heating unit 300 in which a reactant material 304 is printed in a select pattern on a substrate surface 302. Reference numeral 306 indicates the point of ignition for the reactant material layer 304. FIG. 3B shows a side view of the heating unit 300 shown in FIG. 3A, with an adhesive layer 308 overlying the patterned reactant layer 304.

### Igniters

Heating units according to the present invention further comprise at least one igniter to facilitate ignition of the reactant material. Also contemplated are heating units comprising a plurality of igniters. The plurality of igniters helps to ensure complete ignition of all of the reactant material. In one embodiment of the heating units featuring multiple igniters, a plurality of igniters are attached to a single coating of reactant material. In another embodiment, there are multiple coatings of reactant material, each having at least one igniter.

The igniter can comprise any device that is capable of igniting the reactant material to generate a self-sustaining oxidation-reduction reaction. A variety of devices and methods can be used for this purpose, for example and without limitation, optical igniters, percussive igniters, and electrical igniters, as described, for example, in U.S. Patent Publication Nos. 2005/0079166; 2004/0234914; and 2004/0234916.

Alternatively, the igniter can be a printable igniter of the type described in commonly assigned, copending U.S. patent application Ser. No. ______ (Attorney (Attorney Docket No. 84.01R), filed on even date herewith. Such an igniter comprises at least two conductors in a spaced-apart configuration, and a conductive layer bridging the at least two conductors. The conductive layer, which is adapted to initiate and produce a “glow” (i.e., localized heat) upon application of electrical power, has an electrical resistance that is greater than the electrical resistance of both of the at least two conductors. Upon initiation of the conductive layer, heat from the exothermic oxidation of the conductive layer composition is generated sufficient to actuate a reactant composition (e.g., a reactant composition-coated substrate).

Once a portion of the reactant material is ignited, the heat generated by the oxidation-reduction reaction can ignite adjacent unburnt reactant until all of the reactant is consumed in the process of the chemical reaction. The exothermic oxidation-reduction reaction can be initiated by the application of energy to at least a portion of the reactant material. Energy absorbed by the reactant material or by an element in contact with the reactant material can be converted to heat. When the reactant material becomes heated to a temperature above the auto-ignition temperature of the reactants (i.e., the minimum temperature required to initiate or cause self-sustaining combustion in the absence of a combustion source or flame), the oxidation-reduction reaction will initiate, igniting the reactant material in a self-sustaining reaction until the reactant is consumed.

The auto-ignition temperature of a reactant material comprising a metal reducing agent and a metal-containing oxidizing agent as disclosed herein can range from 200° C. to 800° C. In another embodiment, the auto-ignition temperature ranges from 300° C. to 700° C.

While such high auto-ignition temperatures facilitate safe processing and safe use of the reactant material under many use conditions, for example, as a portable medical device, for the same reasons, to achieve such high temperatures, a large amount of energy must be applied to the reactant material to initiate the self-sustaining reaction. Furthermore, the thermal mass represented by the reactant material can require that an impractically high temperature be applied to raise the temperature of the reactant material above the auto-ignition temperature. As heat is being applied to the reactant material and/or a support on which the reactant material is disposed, heat is also being conducted away.

Energy can be applied to ignite the reactant material using a number of methods. For example, a resistive heating element can be positioned in thermal contact with the reactant material which, when a current is applied, can heat the reactant material to the auto-ignition temperature. An electromagnetic radiation source can be directed at the reactant material which, when absorbed, can heat the reactant material to its auto-ignition temperature. An electromagnetic source can include, for example and not by way of limition, lasers, diodes, flashlamps, and microwave sources.

Induction heating can heat the reactant material by applying an alternating magnetic field that can be absorbed by materials having high magnetic permeability, either within the reactant material or in thermal contact with the reactant material. The source of energy can be focused onto the absorbing material to increase the energy density to produce a higher local temperature and thereby facilitate ignition. In certain embodiments, the reactant material can be ignited by percussive forces.

In the pyrotechnic industry, sparks can be used to safely and efficiently ignite reactant compositions. Sparks refer to an electrical breakdown of a dielectric medium or the ejection of burning particles. In the first sense, an electrical breakdown can be produced, for example, between separated electrodes to which a voltage is applied. Sparks can also be produced by ionizing compounds in an intense laser radiation field. Examples of burning particles include those produced by friction and break sparks produced by intermittent electrical current. Sparks of sufficient energy incident on a reactant material can initiate the self-sustaining oxidation-reduction reaction.

When sufficiently heated, the exothermic oxidation-reduction reaction of the reactant material can produce sparks, as well as radiation energy. Thus, in certain embodiments, reliable, reproducible, and controlled ignition of the reactant material can be facilitated by the use of an initiator composition capable of reacting in an exothermic oxidation-reduction reaction. The initiator composition can comprise the same or similar reactants as those comprising the reactant material. In certain embodiments, the initiator composition can be formulated to maximize the production of sparks having sufficient energy to ignite a reactant material. Sparks ejected from an initiator composition can impinge upon the surface of the reactant material, causing the reactant material to ignite in a self-sustaining exothermic oxidation-reduction reaction. The igniter can comprise a physically small, thermally isolated heating element on which is applied a small amount of an initiator composition capable of producing sparks, or the initiator composition can be placed directly on the reactant itself and ignited by a variety of means, including, for example and not by way of limitation, optical, percussive, or electrical igniters.

In certain embodiments, the igniter can comprise a support and an initiator composition disposed on the support. In certain embodiments, the support can be thermally isolated to minimize the potential for heat loss. In this way, dissipation of energy applied to the combination of assembly and support can be minimized, thereby reducing the power requirements of the energy source and facilitating the use of physically smaller and less expensive heat sources. In certain applications, for example, with battery-powered portable medical devices, such considerations can be particularly useful. In certain embodiments, it can be useful that the energy source be a small, low-cost battery, such as a 1.5 V alkaline battery or a 3 V lithium battery. In certain embodiments, the initiator composition can comprise a metal reducing agent and metal-containing oxidizing agent (as broadly defined herein).

The ratio of metal reducing agent to metal-containing oxidizing agent can be selected to determine the appropriate burn and spark-generating characteristics. In certain embodiments, the amount of oxidizing agent in the initiator composition can be related to the molar amount of the oxidizers at or near the eutectic point for the reactant composition. In certain embodiments, the oxidizing agent can be the major component and, in others, the metal reducing agent can be the major component. The particle size of the metal and the metal-containing oxidizer can be varied to determine the burn rate, with smaller particle sizes selected for a faster burn (see, for example, PCT Publication No. WO 2004/01396).

In certain embodiments, an initiator composition can comprise additive materials to facilitate, for example, processing, enhance the mechanical integrity, and/or determine the burn and spark-generating characteristics. The additive materials can be inorganic materials and can function as binding agents, adhesives, gelling agents, thixotropic agents, and/or surfactants. Examples of gelling agents include, but are not limited to, clays such as Laponite® or Cloisite® additives or montmorillonite; metal alkoxides, such as those represented by the formula R—Si(OR)ₙ and M(OR)ₙ, where n can be 3 or 4, and M can be Ti, Zr, Al, B, or another metal; and colloidal particles based on transition metal hydroxides or oxides.

Examples of binding agents include, but are not limited to, soluble silicates, such as Laponite® additives; sodium, potassium, or aluminum silicates; metal alkoxides; inorganic polyanions; inorganic polycations; and inorganic sol-gel materials, such as alumina or silica-based sols. Other useful additive materials include glass beads, diatomaceous earth, nitrocellulose, polyvinylalcohol, guar gum, ethyl cellulose, cellulose acetate, polyvinyl-pyrrolidone, fluoro-carbon rubber (Viton), and other polymers that can function as a binding agent. In certain embodiments, the initiator composition can comprise more than one additive material.

The components of the initiator composition comprising the metal, metal-containing oxidizing agent and/or additive material, and/or any appropriate aqueous- or organic-soluble binding agent can be mixed by any appropriate physical or mechanical method to achieve a useful level of dispersion and/or homogeneity. In certain embodiments, additive materials can be useful in determining certain processing, ignition, and/or burn characteristics of the initiator composition. In certain embodiments, the particle size of the components of the initiator can be selected to tailor the ignition and burn rate characteristics as is known in the art (see, for example, U.S. Pat. No. 5,739,460).

In certain embodiments, an initiator composition can comprise at least one metal, such as those described herein, and at least one oxidizing agent, such as, for example, a chlorate or perchlorate of an alkali metal or an alkaline earth metal or metal oxide and others disclosed herein.

Examples of initiator compositions include compositions comprising 10% Zr: 22.5% B: 67.5% KClO₃; 49.0% Zr: 49.0% MoO₃: 2.0% nitrocellulose; 33.9% Al: 55.4% MoO₃: 8.9% B: 1.8% nitrocellulose; and 26.5% Al: 51.5% MoO₃: 7.8% B: 14.2% Viton (in weight percent).

Other initiator compositions can be used. For example, an initiator composition that can ignite upon application of a percussive force comprises a mixture of sodium chlorate (NaClO₃), phosphorous (P), and magnesium oxide (MgO).

Energy sufficient to heat the initiator composition to the auto-ignition temperature can be applied to the initiator composition and/or the support on which the initiator composition is disposed. The energy source can be any of those disclosed herein, such as resistive heating, radiative heating, inductive heating, optical heating, and percussive heating. In embodiments in which the initiator composition is capable of absorbing the incident energy, the support can comprise a thermally insulating material. In certain embodiments, the incident energy can be applied to a thermally conductive support that can heat the initiator composition above the auto-ignition temperature by thermal conduction.

In certain embodiments, the energy source can be an electrically resistive heating element. The electrically resistive heating element can comprise any material that can maintain integrity at the auto-ignition temperature of the initiator composition. In certain embodiments, the heating element can comprise an elemental metal such as tungsten, an alloy such as nichrome, or other material such as carbon. Materials suitable for resistive heating elements are known in the art. The resistive heating element can have any appropriate form. For example, the resistive heating element can be in the form of a wire, filament, ribbon, or foil. In certain embodiments, the electrical resistance of the heating unit can range from 2Ω to 6Ω. The appropriate resistivity of the heating element can at least in part be determined by the current of the power source, the desired auto ignition temperature, or the desired ignition time. In certain embodiments, the auto-ignition temperature of the initiator composition can range from 200° C. to 800° C. In other embodiments the auto-ignition temperature of the initiator composition can range from 300° C. to 700° C. The resistive heating element can be electrically connected and suspended between two electrodes electrically connected to a power source.

Upon ignition of the reactant material, an exothermic oxidation-reduction reaction produces a considerable amount of energy in a short time, such as for example, in certain embodiments less than 1 second, in certain embodiments less than 500 milliseconds, and in certain embodiments less than 250 milliseconds. Examples of exothermic reactions include electrochemical reactions and metal oxidation-reduction reactions. When used in enclosed heating units, by minimizing the quantity of reactants and the reaction conditions, the reaction can be controlled, but can result in a slow release of heat and/or a modest temperature rise. The temperature rise can exceed 200° C., and in some applications can exceed 250° C. or even 300° C. However, in certain applications, it can be useful to rapidly heat a substrate to temperatures in excess of 200° C. within 1 second or less. Such rapid intense thermal pulses can be useful for vaporizing pharmaceutical compositions to produce aerosols. A rapid intense thermal pulse can be produced using an exothermic oxidation-reduction reaction and, in particular, a thermite reaction involving a metal and a metal-containing oxidizing agent.

When sealed within an enclosure, the exothermic oxidation-reduction reaction can generate a significant increase in pressure. In certain methods of preparation of the heating devices of the invention, the presence of excess air or other gas in the device during fabrication is reduced or eliminated. The presence of excess gas in the heating unit results in an increase in temperature and pressure during ignition of the reactant, which can result in damage to the adhesive layer and/or to the device itself.

The temperature to which one portion of the substrate is heated can be varied with respect to the temperature to which another portion of the substrate is heated in a variety of ways, thereby controlling the rate and/or time of delivery of one or more vaporizable components disposed upon at least a portion of the second surface of the substrate.

Thus, for example, in order to maximize the range of agents which can be heated employing heating units according to the present invention, the ratio of metal reducing agent to metal-containing oxidizing agent can be varied at different locations on the surface of the substrate, thereby providing different temperature maxima at different locations on the surface of the substrate upon ignition of the reactant material. This allows different areas on the surface of the substrate to be exposed to different temperatures, which allows the vaporization of drugs with different heating requirements, optionally at different times.

Similarly, the quantity of reactant material applied to the substrate can be varied at different locations on the first surface of the substrate, so as to achieve different temperature maxima upon ignition of the reactant material.

In any event, it is generally desirable to be able to rapidly heat a portion of the substrate to an elevated temperature (for example, a temperature of at least 200° C.) within, at most, 3 seconds following ignition of the reactant material. In other embodiments, heating of a portion of the substrate to an elevated temperature occurs within 2 seconds, or within 1 second, or even within 0.5 seconds.

### Adhesive Materials

Adhesive materials for use in the present invention should be resistant to cracking, delamination, and/or formation of microchannels upon exposure to elevated temperatures. As used herein, the term “cracking” refers to separation between particles within an adhesive layer, such that the separation would compromise the adhesive surface provided by the adhesive layer. As used herein, the term “delamination” refers to separation (i.e., loss of adhesion) between the adhesive layer and the substrate surface, and/or separation between an adhesive layer and an additional layer(s).

Adhesive materials contemplated for use in the practice of the present invention can be prepared from a variety of materials. The materials may be curable, for example and not by way of limitation, chemically or thermochemically curable. While such curing can be carried out at a variety of temperatures, it is presently preferred that materials employed herein be capable of curing at relatively moderate temperatures, with temperatures below about 300° C., or even below about 225° C.

Adhesive materials contemplated for use herein can be prepared from a variety of materials, including organic or inorganic materials. The amount of organic material present after curing may be minimized, for example within the range of less than about 5% organic material after curing, or even less than about 1% organic material after curing.

The adhesive material can be an organic-based adhesive, an inorganic-based adhesive, or a hybrid organic/inorganic-based adhesive. Inorganic-based adhesives include ceramic materials. Ceramic adhesives suitable for use in the present invention are available, for example and not by way of limitation, from Cotronics Corp. (Brooklyn, NY). Polyester adhesive layers, such as hot melt film 5250 from Bemis Company, Inc. (Shirley, MA) and hot melt film 620 from 3M Company (St. Paul, MN), are also suitable for use in the present invention.

Adhesive materials contemplated for use herein may comprise one or more binding agents and can be prepared, for example, from a combination of a liquid binding agent and an inorganic solid filler material. Examples of binding agents include silicate-based binding agents and phosphate-based binding agents.

Examples of liquid binding agents may comprise a metal silicate (e.g., Mₓ(SiO₄)_{y}) and/or metal phosphate (e.g., Mₓ(PO₄)_{y}) solution, where M is one or more of Li, Na, K, Mg, Ca, Zn, or Al, and x and y are selected so as to satisfy the valence requirements of the respective components of the metal silicate and/or metal phosphate.

Examples of inorganic solid filler materials may comprise metal particles, ceramics, metal oxides, metal silicates, metal phosphates, and metal carbides. Such materials may have melting points above 500° C., with melting points above 1000° C. being desirable in some embodiments. Representative metal particles include stainless steel particles; exemplary metal oxides include alumina, zirconia, silica, magnesia, zinc oxide, kaolin, talc, clay, and combinations thereof; representative ceramics include aluminum nitrides, alumina-silica blends, and zirconium silicates, and combinations thereof; representative metal silicates include zirconium silicate, magnesium silicate, iron silicate, zinc silicate, and combinations thereof; representative metal phosphates include aluminum phosphate, and zirconium phosphate; and representative metal carbides include zinc nitride, zirconium nitride, and combinations thereof.

A wide variety of sizes and shapes are contemplated for solid filler materials employed in the practice of the present invention. For example, such materials may have a particle size of less than about 200 μm in the largest dimension thereof, with particle sizes of no greater than about 100 μm being desirable in some applications. Particles employed in the practice of the present invention can be of any shape, for example and not by way of limitation, spherical, fibrous, cylindrical, coiled, or saddle-shaped, as well as mixtures of different shapes. In certain embodiments, at least 5% of the solid filler includes fibrous particles. In other embodiments, at least 10% of the solid filler includes fibrous particles. In yet other embodiments, at least 25% of the solid filler includes fibrous particles. The inclusion of fibrous particles in the solid filler may improve the crack and/or impact resistance of the solid filler material, which may resulting in improved device performance.

As employed herein, the term “compatible” refers to an adhesive layer that will not significantly compromise the energy generating capacity of the reactant material and will have good adhesion to those surfaces which it contacts (for example, the first substrate, the optionally present second substrate, and the reactant material), etc. It is desirable that the adhesive layer have good adhesive properties with respect to the first substrate (and the second substrate if present) and the reactant material.

In one embodiment of the invention, the adhesive layer can function as an oxidizer, thereby actively participating in the generation of heat. In this manner, the selection of reactant materials can be coordinated with the selection of the material for the adhesive layer.

In some embodiments it may be desirable that, the coefficient of thermal expansion of the adhesive layer be substantially similar to the coefficient of thermal expansion of the substrate.

### Additional Layers

Heating units according to the present invention may optionally comprise at least one additional layer disposed upon at least a portion of the adhesive layer. Such additional layers may be employed to impart a variety of added functions to invention heating units, for example, such additional layers may comprise an insulating layer, may provide gas or moisture impermeable sealing, impact resistance, and/or strong adhesion to the substrate, for example and not by way of limitation.

Such additional layers can be prepared from a variety of materials, for example, organic-based adhesives, inorganic-based adhesives, or hybrid organic/inorganic-based adhesives. Presently preferred materials contemplated for such use include epoxies, silicones, acrylates, polyesters, polyamides, and polyvinyl compounds.

In one embodiment of the heating units of the invention, the adhesive layer comprises a ceramic adhesive, and an additional layer of an epoxy adhesive overlies the ceramic adhesive layer. As ceramic adhesives are often porous, the presence of the epoxy layer allows for hermetic sealing of the unit, which prevents water from seeping into the unit during washing.

Alternatively, the additional layer may comprise a polymeric coating. Polymeric coatings contemplated for use in the invention are substantially impervious to gas, thereby protecting the contents of the invention heating unit from exposure to various atmospheric components which may impact the stability thereof. For example, polymeric coating materials contemplated for use in the practice of the present invention include (meth)acrylate coatings, epoxy coatings, and maleimide-based coatings.

### Drug Supply Units

Heating units according to the present invention may optionally further comprise at least one vaporizable component disposed upon at least a portion of a second surface of the substrate. When the heating unit comprises two substrates in a sandwiched configuration, the heating unit may further comprise at least one vaporizable component disposed upon at least a portion of the second (or outer) surface of the second substrate. Such a configuration allows for the delivery of two different vaporizable components at the same time, one from the outer surface of each substrate.

As readily recognized by those of skill in the art, a wide variety of vaporizable components can be disposed on the heating devices of the invention, and subsequently vaporized. Examples of vaporizable components include physiologically active compounds, industrially important compounds for which vaporization is desirable, and compounds which are useful for a variety of applications when converted into the vapor state, for example, air freshening agents.

In accordance with one embodiment of the present invention, there are provided drug supply units comprising a heating unit as described herein, and at least one drug disposed on at least a portion of a second surface of the substrate.

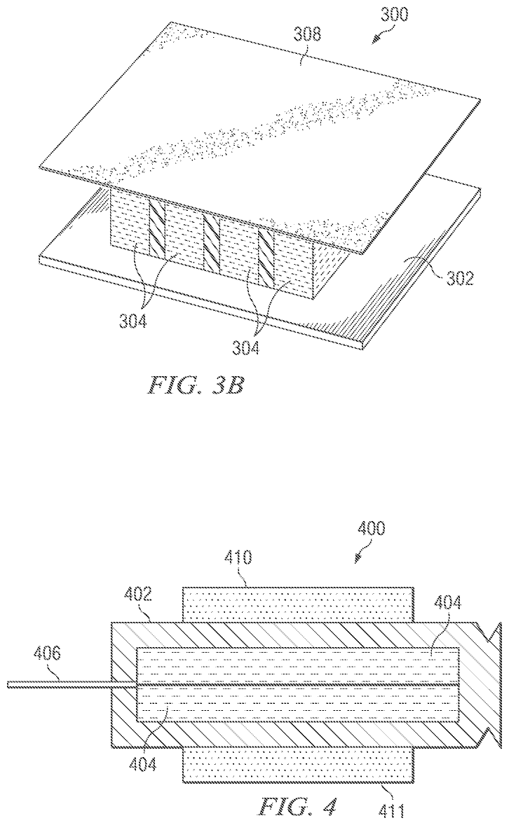

FIG. 4 is a cross-sectional side view of an embodiment of a heating unit described in FIG. 2D which includes an igniter and a drug layer coated onto a substrate surface. The heating unit 400 includes a single substrate 402 folded over itself with a chemical reactant material layer 404 deposited on opposing surfaces of the folded-over substrate 402. The heating unit 400 further includes an igniter 406 shown in contact with reactant layers 404. In other embodiments the igniter need only be in sufficient proximity to, upon ignition, ignite the chemical reactant layer. In this embodiment, the opposing edges of the substrate 402 are seam welded to seal the chemical reactant material layers 244 within the substrate 242. Drug layers 410 and 411 are coated onto the outer (i.e., second) surface of substrate 402. Drug layers 410 and 411 typically comprise the same drug, but may optionally comprise different drugs. Alternatively, one of the layers may comprise a drug, while the other layer comprises a different type of vaporizable component, such as a taste-masking agent.

A variety of drugs can be vaporized for delivery according to the present invention. As used herein, the term “drug” refers to any compound for therapeutic use or non-therapeutic use, including therapeutic agents and substances. As used herein, the term “therapeutic agent” refers to any compound suitable for use in the diagnosis, cure, mitigation, treatment, or prevention of a disease, and any compound used in the mitigation or treatment of symptoms of disease. The term “substances” refers to compounds used for non-therapeutic uses, typically for a recreational or experimental purpose.

Classes of drugs contemplated for use in the practice of the present invention include anesthetics, anticonvulsants, antidepressants, antidiabetic agents, antidotes, antiemetics, antihistamines, anti-infective agents, antineoplastics, antiparkisonian drugs, antirheumatic agents, antipsychotics, anxiolytics, appetite stimulants and suppressants, blood modifiers, cardiovascular agents, central nervous system stimulants, drugs for Alzheimer's disease management, drugs for cystic fibrosis management, diagnostics, dietary supplements, drugs for erectile dysfunction, gastrointestinal agents, hormones, drugs for the treatment of alcoholism, drugs for the treatment of addiction, immunosuppressives, mast cell stabilizers, migraine preparations, motion sickness products, drugs for multiple sclerosis management, muscle relaxants, nonsteroidal anti-inflammatories, opioids, other analgesics and stimulants, opthalmic preparations, osteoporosis preparations, prostaglandins, respiratory agents, sedatives and hypnotics, skin and mucous membrane agents, smoking cessation aids, Tourette's syndrome agents, urinary tract agents, and vertigo agents.

Examples of anesthetic agents include ketamine and lidocaine.

Examples of anticonvulsants include compounds from one of the following classes: GABA analogs, tiagabine, vigabatrin; barbiturates such as pentobarbital; benzodiazepines such as clonazepam; hydantoins such as phenytoin; phenyltriazines such as lamotrigine; miscellaneous anticonvulsants such as carbamazepine, topiramate, valproic acid, and zonisamide.

Examples of antidepressants include amitriptyline, amoxapine, benmoxine, butriptyline, clomipramine, desipramine, dosulepin, doxepin, imipramine, kitanserin, lofepramine, medifoxamine, mianserin, maprotoline, mirtazapine, nortriptyline, protriptyline, trimipramine, venlafaxine, viloxazine, citalopram, cotinine, duloxetine, fluoxetine, fluvoxamine, milnacipran, nisoxetine, paroxetine, reboxetine, sertraline, tianeptine, acetaphenazine, binedaline, brofaromine, cericlamine, olovoxamine, iproniazid, isocarboxazid, moclobemide, phenyhydrazine, pheneizine, selegiline, sibutramine, tranylcypromine, ademetionine, adrafmil, amesergide, amisulpride, amperozide, benactyzine, bupropion, caroxazone, gepirone, idazoxan, metralindole, milnacipran, minaprine, nefazodone, nomifensine, ritanserin, roxindole, Sadenosylmethionine, escitalopran, tofenacin, trazodone, tryptophan, and zalospirone.

Examples of antidiabetic agents include pioglitazone, rosiglitazone, and troglitazone.

Examples of antidotes include edrophonium chloride, flumazenil, deferoxamine, nalmefene, naloxone, and naltrexone.

Examples of antiemetics include alizapride, azasetron, benzquinamide, bromopride, buclizine, chlorpromazine, cinnarizine, clebopride, cyclizine, diphenhydramine, diphenidol, dolasetron, droperidol, granisetron, hyoscine, lorazepam, dronabinol, metoclopramide, metopimazine, ondansetron, perphenazine, promethazine, prochlorperazine, scopolamine, triethylperazine, trifluoperazine, triflupromazine, trimethobenzamide, tropisetron, domperidone, and palonosetron.

Examples of antihistamines include astemizole, azatadine, brompheniramine, carbinoxamine, cetrizine, chlorpheniramine, cinnarizine, clemastine, cyproheptadine, dexmedetomidine, diphenhydramine, doxylamine, fexofenadine, hydroxyzine, loratidine, promethazine, pyrilamine and terfenidine.

Examples of anti-infective agents include compounds selected from one of the following classes: antivirals such as efavirenz; AIDS adjunct agents such as dapsone; aminoglycosides such as tobramycin; antifungals such as fluconazole; antimalarial agents such as quinine; antituberculosis agents such as ethambutol; β-lactams such as cefmetazole, cefazolin, cephalexin, cefoperazone, cefoxitin, cephacetrile, cephaloglycin, cephaloridine; cephalosporins, such as cephalosporin C, cephalothin; cephamycins such as cephamycin A, cephamycin B, and cephamycin C, cephapirin, cephradine; leprostatics such as clofazimine; penicillins such as ampicillin, amoxicillin, hetacillin, carfecillin, carindacillin, carbenicillin, amylpenicillin, azidocillin, benzylpenicillin, clometocillin, eloxacillin, cyclacillin, methicillin, nafcillin, 2-pentenylpenicillin, penicillin N, penicillin O, penicillin S, penicillin V, dicloxacillin; diphenicillin; heptylpenicillin; and metampicillin; quinolones such as eiprofloxacin, clinafloxacin, difloxacin, grepafloxacin, norfioxacin, ofloxacine, temafloxacin; tetracyclines such as doxycycline and oxytetracycline; miscellaneous anti-infectives such as linezolide, trimethoprim and sulfamethoxazole.

Examples of anti-neoplastic agents include droloxifene, tamoxifen, and toremifene.

Examples of anti-parkisonian drugs include amantadine, baclofen, biperiden, benztropine, orphenadrine, procyclidine, trihexyphenidyl, levodopa, carbidopa, andropinirole, apomorphine, benserazide, bromocriptine, budipine, cabergoline, eliprodil, eptastigmine, ergoline, galanthamine, lazabemide, lisuride, mazindol, memantine, mofegiline, pergolide, piribedil, pramipexole, propentofylline, rasagiline, remacemide, ropinerole, selegiline, spheramine, terguride, entacapone, and tolcapone.

Examples of anti-rheumatic agents include diclofenac, hydroxychloroquine and methotrexate.

Examples of antipsychotics include acetophenazine, alizapride, amisuipride, amoxapine, amperozide, aripiprazole, benperidol, benzquinamide, bromperidol, buramate, butaclamol, butaperazine, carphenazine, carpipramine, chlorpromazine, chlorprothixene, clocapramine, clomacran, clopenthixol, clospirazine, clothiapine, clozapine, cyamemazine, droperidol, flupenthixol, fluphenazine, fluspirilene, haloperidol, loxapine, melperone, mesoridazine, metofbnazate, molindrone, olanzapine, penfluridol, pericyazine, perphenazine, pimozide, pipamerone, piperacetazine, pipotiazine, prochiorperazine, promazine, quetiapine, remoxipride, risperidone, sertindole, spiperone, sulpiride, thioridazine, thiothixene, trifluperidol, triflupromazine, trifluoperazine, ziprasidone, zotepine, and zuclopenthixol.

Examples of anxiolytics include alprazolam, bromazepam, oxazepam, buspirone, hydroxyzine, mecloqualone, medetomidine, metomidate, adinazolam, chlordiazepoxide, clobenzepam, flurazepam, lorazepam, loprazolam, midazolam, alpidem, alseroxlon, amphenidone, azacyclonol, bromisovalum, captodiarnine, capuride, carbcloral, carbromal, chloral betaine, eneiprazine, flesinoxan, ipsapiraone, lesopitron, loxapine, methaqualone, methprylon, propanolol, tandospirone, trazadone, zopiclone, and zolpidem.

An example of an appetite stimulant is dronabinol.

Examples of appetite suppressants include fenfluramine, phentermine and sibutramine.

Examples of blood modifiers include cilostazol and dipyridamol.

Examples of cardiovascular agents include benazepril, captopril, enalapril, quinapril, ramipril, doxazosin, prazosin, clonidine, labetolol, candesartan, irbesartan, losartan, telmisartan, valsartan, disopyramide, flecanide, mexiletine, procainaniide, propafenone, quinidine, tocainide, amiodarone, dofetilide, ibutilide, adenosine, gemfibrozil, lovastatin, acebutalol, atenolol, bisoprolol, esmolol, metoprolol, nadolol, pindolol, propranolol, sotalol, diltiazem, nifedipine, verapamil, spironolactone, bumetanide, ethacrynic acid, furosemide, torsemide, amiloride, triamterene, and metolazone.

Examples of central nervous system stimulants include amphetamine, brucine, caffeine, dexfenfluramine, dextroamphetamine, ephedrine, fenfluramine, mazindol, methyphenidate, pemoline, phentermine, sibutramine, and modafinil.

Examples of drugs for Alzheimer's disease management include donepezil, galanthamine and tacrin.

Examples of drugs for cystic fibrosis management include ciprofloxacin, 3-isobutyl-1-methylxanthine, XAC and analogues; 4-phenylbutyric acid; genistein and analogous isoflavones; and milrinone.

Examples of diagnostic agents include adenosine and and aminohippuric acid.

Examples of dietary supplements include melatonin and vitamin-E.

Examples of drugs for erectile dysfunction include tadalafil, sildenafil, vardenafil, apomorphine, apomorphine diacetate, phentolamine, and yohimbine.

Examples of gastrointestinal agents include loperamide, atropine, hyoscyamine, famotidine, lansoprazole, omeprazole, and rebeprazole.

Examples of hormones include: testosterone, estradiol, and cortisone.

Examples of drugs for the treatment of alcoholism include naloxone, naltrexone, and disulfiram.

An examples of a drug for the treatment of addiction is buprenorphine.

Examples of immunosupressives include mycophenolic acid, cyclosporin, azathioprine, tacrolimus, and rapamycin.

Examples of mast cell stabilizers include cromolyn, pemirolast, and nedocromil.

Examples of drugs for migraine headache include almotriptan, alperopride, codeine, dihydroergotamine, ergotamine, eletriptan, frovatriptan, isometheptene, lidocaine, lisuride, metoclopramide, naratriptan, oxycodone, propoxyphene, rizatriptan, sumatriptan, tolfenamic acid, zolmitriptan, amitriptyline, atenolol, clonidine, cyproheptadine, diltiazem, doxepin, fluoxetine, lisinopril, methysergide, metoprolol, nadolol, nortriptyline, paroxetine, pizotifen, pizotyline, propanolol, protriptyline, sertraline, timolol, and verapamil.

Examples of motion sickness products include diphenhydramine, promethazine, and scopolamine.

Examples of drugs for multiple sclerosis management include bencyclane, methylprednisolone, mitoxantrone, and prednisolone.

Examples of muscle relaxants include baclofen, chlorzoxazone, cyclobenzaprine, methocarbamol, orphenadrine, quinine, and tizanidine.

Examples of nonsteroidal anti-inflammatory drugs include aceclofenac, acetaminophen, alminoprofen, amfenac, aminopropylon, amixetrine, aspirin, benoxaprofen, bromfenac, bufexamac, carprofen, celecoxib, choline, salicylate, cinchophen, cinmetacin, clopriac, clometacin, diclofenac, diflunisal, etodolac, fenoprofen, flurbiprofen, ibuprofen, indomethacin, indoprofen, ketoprofen, ketorolac, mazipredone, meclofenamate, nabumetone, naproxen, parecoxib, piroxicam, pirprofen, rofecoxib, sulindac, tolfenamate, tolmetin, and valdecoxib.

Examples of opioid drugs include alfentanil, allylprodine, alphaprodine, anileridine, benzylmorphine, bezitramide, buprenorphine, butorphanol, carbiphene, cipramadol, clonitazene, codeine, dextromoramide, dextropropoxyphene, diamorphine, dthydrocodeine, diphenoxylate, dipipanone, fentanyl, hydromorphonc, L-alpha acetyl methadol, lofentanil, levorphanol, meperidine, methadone, meptazinol, metopon, morphine, nalbuphine, nalorphine, oxycodone, papaveretum, pethidine, pentazocine, phenazocine, remifentanil, sufentanil, and tramadol.

Examples of other analgesic drugs include apazone, benzpiperylon, benzydramine, caffeine, clonixin, ethobeptazine, flupirtine, nefopam, orphenadrine, propacetamol, and propoxyphene.

Examples of opthalmic preparation drugs include ketotifen and betaxolol.

Examples of osteoporosis preparation drugs alendronate, estradiol, estropitate, risedronate and raloxifene.

Examples of prostaglandin drugs include epoprostanol, dinoprostone, misoprostol, and alprostadil.

Examples of respiratory agents include albuterol, ephedrine, epinephrine, fomoterol, metaproterenol, terbutaline, budesonide, ciclesonide, dexamethasone, flunisolide, fluticasone propionate, triamcinolone acetonide, ipratropium bromide, pseudoephedrine, theophylline, montelukast, zafirlukast, ambrisentan, bosentan, enrasentan, sitaxsentan, tezosentan, iloprost, treprostinil, and pirfenidone.

Examples of sedative and hypnotic drugs include butalbital, chlordiazepoxide, diazepam, estazolam, flunitrazepam, flurazepam, lorazepam, midazolam, temazepam, triazolam, zaleplon, zolpidem, and zopiclone.

Examples of skin and mucous membrane agents include isotretinoin, bergapten and methoxsalen.

Examples of smoking cessation aids include nicotine and varenicline.

An example of a Tourette's syndrome agent includes pimozide.

Examples of urinary tract agents include tolteridine, darifenicin, propantheline bromide, and oxybutynin.

Examples of vertigo agents include betahistine and indolizine.

In certain embodiments, a drug can further comprise substances to enhance, modulate, and/or control release, aerosol formation, intrapulmonary delivery, therapeutic efficacy, therapeutic potency, and/or stability of the drug. For example, to enhance therapeutic efficacy, a drug can be co-administered with one or more active agents to increase the absorption or diffusion of the first drug through the pulmonary alveoli, or to inhibit degradation of the drug in the systemic circulation. In certain embodiments, a drug can be co-administered with active agents having pharmacological effects that enhance the therapeutic efficacy of the drug. In certain embodiments, a drug can comprise compounds that can be used in the treatment of one or more diseases, conditions, or disorders. In certain embodiments, a drug can comprise more than one compound for treating a disease, condition, or disorder, or for treating more than one disease, condition, or disorder.

A film of drug can be applied to the substrate by any appropriate method, depending on such factors as the physical properties of the specific drug and the thickness of the film, among others. In certain embodiments, methods of applying a drug to the exterior substrate surface include, but are not limited to, brushing, dip coating, spray coating, screen printing, roller coating, inkjet printing, vapor-phase deposition, and spin coating. In certain embodiments, the drug can be prepared as a solution comprising at least one solvent and applied to the exterior surface. In certain embodiments, a solvent can comprise a volatile solvent such as, for example, but without limitation, acetone or isopropanol. In certain embodiments, the drug can be applied to the exterior surface of the substrate as a melt. In certain embodiments, the drug can be applied to a support having a release coating and transferred to a substrate from the support. For drugs that are liquid at room temperature, thickening agents can be admixed with the drug to produce a viscous composition comprising the drug that can be applied to the exterior substrate surface by any appropriate method, including those described herein. In certain embodiments, a film of compound can be formed during a single application or can be formed during repeated applications to increase the final thickness of the film. In certain embodiments, the final thickness of a film of drug disposed on the exterior substrate surface can be less than 50 μm; in certain embodiments, less than 20 μm; in certain embodiments, less than 10 μm; in certain embodiments, within the range of 0.1 μm to 10 μm.

In certain embodiments, the film can comprise a therapeutically effective amount of at least one drug. Therapeutically effective amount refers to an amount sufficient to affect treatment when administered to a patient or user in need of treatment. Treating or treatment of a disease, condition, or disorder refers to arresting or ameliorating; reducing the risk of acquiring; reducing the development of, or at least one of the clinical symptoms of; or reducing the risk of developing, or at least one of the clinical symptoms of, a disease, condition, or disorder. Treating or treatment also refers to inhibiting the disease, condition, or disorder, either physically (e.g., stabilization of a discernible symptom), physiologically (e.g., stabilization of a physical parameter), or both, and inhibiting at least one physical parameter that may not be discernible to the patient. Further, treating or treatment refers to delaying the onset of the disease, condition, or disorder, or at least symptoms thereof, in a patient which may be exposed to or predisposed to a disease, condition, or disorder, even though that patient does not yet experience or display symptoms of the disease, condition, or disorder.

In certain embodiments, the drug film can comprise one or more pharmaceutically acceptable carriers, adjuvants, and/or excipients. As used herein, the term “pharmaceutically acceptable” refers to a substance that is approved or approvable by a regulatory agency of the federal government or a state government, or listed in the U.S. Pharmacopoeia or other generally recognized pharmacopoeia for use in animals, and more particularly, in humans.

The drug can be applied to the substrate surface using any appropriate method, such as for example, brushing, dip coating, screen printing, roller coating, spray coating, inkjet printing, stamping, and vapor deposition. The drug can also be applied to a support having a release layer and transferred to the substrate. The drug can be suspended in a volatile solvent such as, for example, but not limited to, acetone or isopropanol to facilitate application to the substrate. A volatile solvent can be removed at room temperature or at elevated temperature, with or without application of a reduced pressure. In certain embodiments, the solvent can comprise a pharmaceutically acceptable solvent. In certain embodiments, residual solvent can be reduced to a pharmaceutically acceptable level.

The drug can be disposed on the substrate in any appropriate form such as a solid, viscous liquid, liquid, crystalline solid, or powder. In certain embodiments, the film of drug can be crystallized after disposition on the substrate.

In one aspect, the second surface of the above-described drug supply unit may have a plurality of portions, such that different drugs can be disposed on different portions, thereby facilitating delivery of different drugs from the same device and/or the delivery of drugs in specified sequence.

The above-described drug supply units facilitate producing an aerosol of a drug. This can be readily accomplished by initiating an exothermic reaction of the reactant material of the above-described drug supply unit, thereby vaporizing the drug. Thus, a drug supply unit according to the present invention is configured such that the reactant material heats a portion of the exterior surface of the substrate to a temperature sufficient to thermally vaporize the drug, in certain embodiments within 3 seconds following ignition of the reactant material, in other embodiments within 1 second following ignition of the reactant material, in other embodiments within 800 milliseconds following ignition of the reactant material, in other embodiments within 500 milliseconds following ignition of the reactant material, and in other embodiments within 250 milliseconds following ignition of the reactant material.

In certain embodiments, a drug supply unit can generate an aerosol comprising a drug that can be inhaled directly by a user and/or can be mixed with a delivery vehicle, such as a gas, to produce a stream for delivery (for example, via a spray nozzle) to a topical site for a variety of treatment regimens, including acute or chronic treatment of a skin condition, administration of a drug to an incision site during surgery, or to an open wound.

In certain embodiments, rapid vaporization of a drug film can occur with minimal thermal decomposition of the drug. For example, in certain embodiments, less than 10% of the drug is decomposed during thermal vaporization, and in certain embodiments, less than 5% of the drug is decomposed during thermal vaporization. In certain embodiments, a drug can undergo a phase transition to a liquid state and then to a gaseous state, or can sublime (i.e., pass directly from a solid state to a gaseous state).

In certain embodiments, a drug can include a pharmaceutical compound. In certain embodiments, the drug can include a therapeutic compound or a non-therapeutic compound. A non-therapeutic compound refers to a compound that can be used for recreational, experimental, or pre-clinical purposes.

The above-described drug supply units also facilitate delivering a drug to a patient in need thereof. This can be readily accomplished by administering a therapeutically effective amount of a drug to a patient in the form of an aerosol, where the aerosol is produced by the above-described method of producing an aerosol.

In accordance with still another embodiment of the present invention, there are also provided drug delivery devices which include a heating unit as described herein, at least one drug disposed on at least a portion of the second surface of the substrate, and an enclosure therefore, including a conduit for delivery of vaporized drug to a subject in need thereof.

The above-described drug delivery devices can be employed for preparation of an aerosol of a drug. This can be readily accomplished by initiating an exothermic reaction of the reactant material of the above-described drug delivery devices, thereby vaporizing the drug.

Similarly, the above-described drug delivery devices can be employed for delivering a drug to a patient in need thereof. Such delivery is accomplished by administering a therapeutically effective amount of drug to the patient in the form of an aerosol, where the aerosol is produced by the above-described method.

Drug delivery devices of the invention may further comprise a housing defining an airway, a heating unit as disclosed herein, a drug disposed on a portion of the exterior surface of a substrate of the heating unit, where the portion of the exterior surface comprising the drug is configured to be disposed within the airway, and an initiator configured to ignite the reactant material. Drug delivery devices can incorporate the heating units and drug supply units disclosed herein.

The drug delivery device can comprise a housing defining an airway. The housing can define an airway having any appropriate shape or dimensions, and can comprise at least one inlet and at least one outlet. The dimensions of an airway can at least in part be determined by the volume of air that can be inhaled through the mouth or the nostrils by a user in a single inhalation, the intended rate of airflow through the airway, and/or the intended airflow velocity at the surface of the substrate that is coupled to the airway and on which a drug is disposed.

In certain embodiments, airflow can be generated by a patient inhaling with the mouth on the outlet of the airway, and/or by inhaling with the nostrils on the outlet of the airway. In certain embodiments, airflow can be generated by injecting air or a gas into the inlet such, as for example, by mechanically compressing a flexible container filled with air and/or gas, or by releasing pressurized air and/or gas into the inlet of the airway. Generating an airflow by injecting air and/or gas into the airway can be useful in drug delivery devices intended for topical administration of an aerosol comprising a drug.

In certain embodiments, a housing can be dimensioned to provide an airflow velocity through the airway sufficient to produce an aerosol of a drug during thermal vaporization. In certain embodiments, the airflow velocity can be at least 1 m/sec in the vicinity of the substrate on which the drug is disposed.

In certain embodiments, a housing can be dimensioned to provide a certain airflow rate through the airway. In certain embodiments, the airflow rate through the airway can range from 5 L/min to 120 L/min. In certain embodiments, an airflow rate within the range of 5 L/min to 120 L/min can be produced during inhalation by a user when the outlet exhibits a cross-sectional area within the range of 0.1 cm² to 20 cm². In certain embodiments, the cross-sectional area of the outlet can be within the range of 0.5 cm² to 5 cm², and in certain embodiments, within the range of 1 cm² to 2 cm².

In certain embodiments, an airway can comprise one or more airflow control valves to control the airflow rate and airflow velocity in the airway. In certain embodiments, an airflow control valve can comprise, but is not limited to, at least one valve such as an umbrella valve, a reed valve, a flapper valve, or a flapping valve that bends in response to a pressure differential. In certain embodiments, an airflow control valve can be located at the outlet of the airway, at the inlet of the airway, within the airway, and/or can be incorporated into the walls of a housing defining the airway. In certain embodiments, an airflow control valve can be actively controlled, for example, can be activated electronically such that a signal provided by a transducer located within the airway can control the position of the valve, or passively controlled, such as, for example, by a pressure differential between the airway and the exterior of the device.

### Method of Making

In accordance with another embodiment of the present invention, there are provided methods for making heating units, as described herein. Such methods include applying a reactant material surface of a to a substrate, where the reactant material is capable of undergoing an exothermic reaction, and thereafter applying an aqueous slurry of an adhesive to form at least one layer of adhesive on top of the reactant material. The adhesive layer(s) can include one or more high temperature inorganic adhesives and can withstand temperatures of at least 300° C. A first layer of adhesive is compatible with the reactant material and the first layer of adhesive adheres to the substrate and, optionally, to the reactant material. Any subsequent layer of adhesive adheres to the preceding layer of adhesive, and at least the final layer of adhesive is resistant to cracking and/or delamination upon exposure to elevated temperatures, e.g., in some embodiments, at least 100° C., in other embodiments, at least 200° C., in other embodiments, at least 300° C.

The adhesive may be in the form of a slurry (e.g., an aqueous slurry or a nonaqueous slurry such as an inorganic slurry). When the adhesive is in the form of an aqueous slurry, heating the resulting heating unit under conditions suitable to remove a substantial amount of the water is recommended. In certain embodiments, heating of the resulting article removes at least 25% of the water. In other embodiments, heating of the resulting article removes at least 50% of the water. In other embodiments, heating of the resulting article removes at least 75% of the water. In yet other embodiments, heating of the resulting article removes at least 90% of the water.

As readily recognized by those of skill in the art, conditions suitable to remove substantially all of the water from the above-described article include heating to a temperature within the range of about 50° C. to about 300° C. for a time period within the range of about 0.5 hour up to about 24 hours, or longer.

Optionally, the resulting article can be subjected to additional heating sufficient to cure the adhesive layer. As readily recognized by those of skill in the art, conditions suitable to carry out such heat curing of the above-described article include heating to a temperature within the range of about 50° C. to about 300° C. for a time period within the range of about 0.25 hour up to about 12 hours, or longer.

Heating units according to the present invention can optionally be hermetically sealed. Such articles would have excellent long-term storage properties, as exposure to moisture and air would be prevented. Hermetic sealing of the heating units can be readily performed using techniques and materials that are known in the art.

Sealing of the heating units can also be accomplished using a number of well-known methods, such as, for example and not by way of limitation, seam welding, spot welding, crimping, molding, and ultrasonic welding, according to techniques known in the art. One or more of the above or other methods can be employed.

### Method of Delivering a Drug to a Patient

In accordance with still another embodiment of the present invention, there are provided methods of delivering a drug to a patient in need thereof employing drug supply units of the invention.

Certain embodiments include methods of producing an aerosol of a compound using the heating units, drug supply units, and drug delivery devices disclosed herein. In certain embodiments, the aerosol produced by an apparatus can comprise a therapeutically effective amount of a drug. The temporal and spatial characteristics of the heat applied to thermally vaporize the compound disposed on the substrate and the airflow rate can be selected to produce an aerosol comprising a drug having certain characteristics. For example, for intrapulmonary delivery, it is known that aerosol particles having a mean mass aerodynamic diameter within the range of 0.01 μm to 0.1 μm, or within the range of 1 μm to 3.5 μm, can facilitate efficient transfer of drugs from alveoli to the systemic circulation. In applications in which the aerosol is applied topically, the aerosol can have the same or different characteristics.

Certain embodiments include methods of treating a disease in a patient in need of such treatment comprising administering to the patient an aerosol comprising a therapeutically effective amount of a drug, where the aerosol is produced by the methods and devices disclosed herein. The aerosol can be administered by inhalation through the mouth, by nasal ingestion, and/or by topical application.

### EXAMPLES

### Example One: Preparation of Reactant Coating Formulation

Various reactant coating formulations were prepared by adjusting the weight percentages of the components. The reactant coating formulation was prepared by homogeneously mixing 60-80% Zr (ca. 3.0 μm, Chemetall, Frankfurt, Germany), 20-40% Fe₂O₃ (ca.1.0 μm, Elementis, East St. Louis, IL) with 1-10% of Laponite® additive (Southern Clay Products, Gonzales, TX) in water, using a Thinky® mixer (Tokyo, Japan). After thorough mixing, the slurry was transferred to a syringe reservoir and allowed to sit for at least 6 hours before coating onto stainless steel foil substrates using an automated tip dispenser (Intelligent Actuators, Torrance, CA).

### Example Two: Preparation of Single Substrate Heat Units

A 304 stainless steel substrate (obtained from Brown Metals Company, Rancho Cucamonga, CA) was coated with the reactant formulation prepared as described in Example One, above, then further overcoated with the ceramic adhesive Durabond™ 954 (manufactured by Cotronics Corp., Brooklyn, NY) along with an igniter (as shown in FIG. 1A). The ceramic adhesive was cured according to vendor specifications.

The heating units of the invention are designed to withstand the heat produced during ignition of the device. Typically, only a small area of the layer of reactant material is exposed and in contact with the igniter. Inspection of the heat unit before and after ignition of the reactant formulation revealed that the heat unit retained its integrity after activation. Infrared thermal imaging analysis of the heat unit using an infrared thermal imaging camera (FLIR Systems, Inc., North Billerica, MA) during ignition of discrete portions of a heat unit having multiple areas containing reactant materials (each such are being referred to herein as a “cell”) showed uniform distribution of heat across the various cells when ignited. Cells which were not ignited did not show any production of heat.

### Example Three: Preparation of Dual Substrate Heat Units

A T430 stainless steel substrate (obtained from Precision Metals Corp., Bay Shore, NY) was coated with the reactant formulation prepared as described in Example One, above, then further overcoated with the ceramic adhesive 3000° F. Resbond™ 989 (manufactured by Cotronics Corp. Brooklyn, NY) along with an igniter, then further bonded to a blank steel substrate (as shown in FIG. 1B). The ceramic adhesive was cured according to vendor specifications.

Inspection of the heat unit before and after ignition of the reactant formulation revealed that the heat unit retained its integrity after activation. Infrared thermal imaging analysis of the device using an infrared thermal imaging camera (FLIR Systems, Inc., North Billerica, MA) during ignition of various reactant cells showed uniform distribution of heat across the various cells when ignited. Cells which were not ignited did not show any production of heat.

### Example Four: Preparation of Double-Sided Heat Units

The surfaces of two 304 stainless steel foils (obtained from Brown Metals Company, Rancho Cucamonga, CA) were coated with the reactant formulation prepared as described in Example One, above. The two steel foils were then bonded face-to-face (i.e., reactant coating-to-reactant coating, as shown in FIG. 2B) using the ceramic adhesive Pyro-Putty® 1000 (manufactured by Aremco Products, Inc., Valley Cottage, NY), while inserting an electrical initiator that generates localized heat or spark. The ceramic adhesive was cured according to vendor specifications. The edges of the heat unit were further hermetically sealed using UV-curable epoxy 1128-M (manufactured by DYMAX Corporation, Torrington, CT).

Inspection of the heat unit before and after ignition of the reactant formulation revealed that the heat unit retained its integrity after activation. Infrared thermal imaging analysis of the device using an infrared thermal imaging camera (FLIR Systems, Inc., North Billerica, MA) during ignition of various reactant cells showed uniform distribution of heat across the various cells when ignited. Cells which were not ignited did not show any production of heat.

It is to be understood that, while the invention has been described in conjunction with the preferred specific embodiments thereof, that the description above as well as the examples that follow are intended to illustrate and not limit the scope of the invention. The practice of the present invention will employ, unless otherwise indicated, conventional techniques of chemistry, manufacturing and engineering, and the like, which are within the skill of the art. Other aspects, advantages and modifications within the scope of the invention will be apparent to those skilled in the art to which the invention pertains. Such techniques are explained fully in the literature.

All patents, patent applications, and publications mentioned herein, both supra and infra, are hereby incorporated by reference.

Various embodiments of the disclosure could also include permutations of the various elements recited in the claims as if each dependent claim was multiple dependent claim incorporating the limitations of each of the preceding dependent claims as well as the independent claims. Such permutations are expressly within the scope of this disclosure.

What is claimed is:

1. A heating unit comprising:
a substrate having a first surface and a second surface;
a chemical reactant material capable of undergoing an exothermic reaction disposed upon at least a portion of the first surface of the substrate;
an igniter in proximity with the chemical reactant material; and
a layer of an adhesive material overlying at least a portion of at least one of the chemical reactant material and the first surface of the substrate.

2. The heating unit according to claim 1, wherein the adhesive layer overlies at least a portion of the reactant material, and wherein the adhesive material is compatible with the reactant material.

3. The heating unit according to claim 1, wherein the heating unit further comprises a second substrate having a first surface and a second surface.

4. The heating unit according to claim 3, wherein the first surface of the second substrate is in contact with the adhesive layer.

5. The heating unit according to claim 3, wherein a chemical reactant material capable of undergoing an exothermic reaction is disposed on at least a portion of the first surface of the second substrate, and wherein the chemical reactant material disposed on at least a portion of the first surface of the second substrate is in contact with the adhesive layer.

6. The heating unit according to claim 3, wherein the first and second substrates are part of a single component, folded together and sealed so as to form a unitary structure containing the reactant material within.

7. The heating unit according to claim 1, wherein the substrate comprises a metal foil.

8. The heating unit according to claim 1, wherein the chemical reactant material comprises a metal reducing agent and a metal-containing oxidizing agent.

9. The heating unit according to claim 8, wherein the chemical reactant material further comprises a binding agent.

10. The heating unit according to claim 8, wherein the chemical reactant material is selected from the group consisting of: Zr:Fe₂O₃, Zr:Fe₂O₃:MnO₂, Zr:MoO₃, Zr:CuO, Zr:Co₃O₄, and Zr:Co₂O₃.

11. The heating unit according to claim 10, wherein the chemical reactant material further comprises Laponite® as a binding agent.

12. The heating unit according to claim 1, wherein the igniter is a printable igniter comprising:
a) at least two conductors in a spaced-apart configuration; and
b) an electrically conductive layer bridging the at least two conductors, wherein the conductive layer has an electrical resistance greater than the electrical resistance of the at least two conductors.

13. The heating unit according to claim 1, wherein the adhesive material is selected from the group consisting of: a ceramic adhesive, an inorganic adhesive, an organic adhesive, and an organic/inorganic composite adhesive.

14. The heating unit according to claim 13, wherein the adhesive material is curable at a temperature within the range of 60° C. to 400° C.

15. The heating unit according to claim 1, wherein the heating unit further comprises an additional layer of material overlying the adhesive layer.

16. The heating unit according to claim 15 wherein the additional layer of material overlaying the adhesive layer is substantially impermeable to gas and liquids.

17. The heating unit according to claim 1, wherein a vaporizable component comprising a drug is coated onto the second surface of the substrate.
